(19) United States
(12) Patent Application Publication
(10) Pub. No.: US 2017/0363528 A1
(43) Pub. Date: Dec. 21, 2017

(54) Resin Flow Analysis Method and Non-Transitory Computer-Readable Recording Medium

(71) Applicants: TORAY ENGINEERING CO., LTD., Tokyo (JP); HONDA MOTOR CO., LTD., Tokyo (JP)

(72) Inventors: Ryo NAKANO, Kyoto-shi (JP); Yuji OKADA, Kyoto-shi (JP); Daisuke URAKAMI, Tokyo (JP); Katsuya SAKABA, Tokyo (JP); Masatoshi KOBAYASHI, Wako-shi (JP); Koji DAN, Wako-shi (JP)

(21) Appl. No.: 15/627,675

(22) Filed: Jun. 20, 2017

(30) Foreign Application Priority Data
Jun. 21, 2016 (JP) 2016-122712

(51) Int. Cl. G01N 11/00; G01N 33/44; G01L 19/00

(52) U.S. Cl. CPC G01N 11/00; G01L 19/0092; G01N 33/442

(57) ABSTRACT

A resin flow analysis method includes dividing a mold space model into small elements, acquiring a penetration coefficient, acquiring a flow conductance, and performing flow analysis of a resin in each of the small elements in the mold space model based on a first relational expression of the small elements of a base material portion relating to the penetration coefficient and a second relational expression of the small elements of a space portion relating to the flow conductance.

### CROSS-REFERENCE TO RELATED APPLICATION

The priority application number JP2016-122712, Resin Flow Analysis Method, Program, and Computer-Readable Recording Medium, Jun. 21, 2016, Ryo Nakano, Yuji Okada, Daisuke Urakami, Katsuya Sakaba, Masatoshi Kobayashi, and Koji Dan, upon which this patent application is based, is hereby incorporated by reference.

### BACKGROUND OF THE INVENTION

### Field of the Invention

The present invention relates to a resin flow analysis method and a non-transitory computer-readable recording medium that records a program.

### Description of the Background Art

A resin flow analysis method is known in general. Such a resin flow analysis method is disclosed in Japanese Patent Laying-Open No. 2003-011170, for example.

The aforementioned Japanese Patent Laying-Open No. 2003-011170 discloses a method for analyzing the flow behavior of a resin that penetrates into a base material in a mold in RTM (resin transfer molding) in which a compound material is molded by arranging the base material such as continuous fiber arranged in a sheet form in the mold, injecting the resin into the mold, and causing the resin to penetrate into the base material. In the aforementioned Japanese Patent Laying-Open No. 2003-011170, the inside of the mold is divided into small elements, and analysis is performed based on the Darcy's law in which the flow behavior of the resin in each of the small elements is expressed as a function of pressure.

In the RTM, the base material arranged in a sheet form is not necessarily tightly filled in the mold, and a space portion in which no base material is arranged is formed between the base material and an inner wall surface of the mold or in a portion in which the base material overlaps with another base material, for example. Furthermore, a region inside the mold in which no base material is arranged may be intentionally designed to be provided. In addition, a molding technology called compression RTM in which after a base material is arranged in a mold, a space portion is provided by opening the mold to a certain degree in advance, a resin is injected, the mold is thereafter closed, and the resin is caused to penetrate into the base material may be used. When flow analysis of the resin is performed in the case where the base material portion and the space portion exist in the mold, it is necessary to deal with a phenomenon in which the resin penetrates (flows) simultaneously into both the base material and the space portion.

As a common method for analyzing the flow in the space portion, Stokes' approximation is performed on the flow, and the flow behavior of the resin can be analyzed as a function of pressure and velocity (Stokes' approximation formula) in each of the small elements by assuming from the momentum conservation law that the influences of gravity and inertia are small, for example.

However, in the flow analysis in the case where both the base material portion and the space portion exist in the RTM, the analysis method using the Stokes' approximation formula is applied to the space portion, and the analysis method based on the Darcy's law as described in the aforementioned Japanese Patent Laying-Open No. 2003-011170 is applied to the base material portion, and hence when the flows of the resin in the space portion and the base material portion are collectively solved, it is difficult to deal with a boundary between the space portion and the base material portion due to different forms of functions. More specifically, there are four variables of a pressure and direction components of a velocity in the case where the Stokes' approximation formula is used whereas there is one variable of a pressure in the function based on the Darcy's law. Thus, computation for dealing with the boundary portion is further required, and the computation load is disadvantageously increased (or the processing time is increased).

A method using the Darcy-Brinkman equation in which both the space portion and the base material portion are expressed is proposed, but in the method, the computational result is not stabilized, and very massive computation is required, and hence the method is disadvantageously unpractical.

### SUMMARY OF THE INVENTION

The present invention has been proposed in order to solve the aforementioned problems, and an object of the present invention is to provide a resin flow analysis method by which a base material portion and a space portion can be collectively stably analyzed at a high speed while the amount of computation is reduced even when both the base material portion and the space portion exist in RTM and a non-transitory computer-readable recording medium.

In order to attain the aforementioned object, a resin flow analysis method according to a first aspect of the present invention is a resin flow analysis method for performing flow analysis of a resin injected into a mold using a mold space model that includes a base material portion made of continuous fiber or a porous body arranged in a sheet form and a space portion in which the base material portion is not arranged, and includes dividing the mold space model into small elements, acquiring a penetration coefficient that represents penetration characteristics of the resin into the base material portion, acquiring a flow conductance that represents flow characteristics of the resin in the space portion, and performing the flow analysis of the resin in each of the small elements in the mold space model based on a first relational expression of the small elements of the base material portion relating to the penetration coefficient, a viscosity of the resin, and a pressure and a second relational expression of the small elements of the space portion relating to the flow conductance, the viscosity of the resin, and the pressure. In this description, the penetration coefficient represents readiness with which a fluid (resin) penetrates into the base material portion, and as the penetration coefficient is increased, the fluid (resin) more readily penetrates. The flow conductance represents readiness with which the fluid (resin) flows, and as the flow conductance is increased, the fluid (resin) more readily flows.

As hereinabove described, the resin flow analysis method according to the first aspect of the present invention includes the performing of the flow analysis of the resin in each of the small elements in the mold space model based on the first relational expression of the small elements of the base material portion relating to the penetration coefficient, the viscosity of the resin, and the pressure and the second relational expression of the small elements of the space portion relating to the flow conductance, the viscosity of the resin, and the pressure. Thus, the penetration coefficient, the viscosity of the resin, and the flow conductance are acquired in advance such that the flow analysis of the resin can be performed on the base material portion and the space portion with the first relational expression and the second relational expression that contain the pressure as a common variable. The space portion and the base material portion can be expressed by the relational expressions having the common variable (pressure), and hence the base material portion and the space portion can be collectively stably analyzed at a high speed while the amount of computation is reduced, unlike the case where different variables are used for the space portion and the base material portion as the conventional art. Furthermore, when the number of variables varies depending on the space portion and the base material portion, the amount of computation of the entire mold space is mainly determined by one (space portion) using a larger number of variables, and is exponentially increased as the number of variables is increased. Thus, the first relational expression and the second relational expression, both of which use the pressure as a common variable, are used such that the number of used variables can be reduced, and hence the amount of computation can be reduced. Consequently, even when both the base material portion and the space portion exist in RTM, the base material portion and the space portion can be collectively stably analyzed at a high speed while the amount of computation is reduced.

In the aforementioned resin flow analysis method according to the first aspect, the first relational expression and the second relational expression each are preferably expressed by a common relational expression containing a coefficient that represents the penetration characteristics or the flow characteristics of the resin, and in the performing of the flow analysis, the penetration coefficient is preferably applied as the coefficient of the common relational expression for the small elements of the base material portion, and the flow conductance is preferably applied as the coefficient for the small elements of the space portion. According to this structure, the base material portion and the space portion can be analyzed by the same relational expressions, the coefficient parts of which are different, and hence a boundary portion can be continuously dealt with, and the base material portion and the space portion can be collectively and stably analyzed.

In the aforementioned resin flow analysis method according to the first aspect, the first relational expression is preferably a following expression (1), and the second relational expression is preferably a following expression (2):

$$\frac{K}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 \qquad ( 1 )$$

where K represents a penetration coefficient tensor, η represents the viscosity of the resin, x, y, and z represent positions of the small elements, and P represents a pressure of each of the small elements, and

$$\frac{c}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 \qquad ( 2 )$$

where c represents the flow conductance, η represents the viscosity of the resin, x, y, and z represent the positions of the small elements, and P represents the pressure of each of the small elements. According to this structure, the flow analysis can be performed on the base material portion and the space portion by the relational expressions in which all except the coefficient parts (the penetration coefficient and the flow conductance) are in common. Consequently, only the coefficient part (the penetration coefficient or the flow conductance) varies according to whether the small element of interest belongs to the base material portion or the space portion, and the entire mold space can be easily analyzed.

In the aforementioned resin flow analysis method according to the first aspect, in the acquiring of the flow conductance, a value that varies according to a flow direction of the resin, which is a direction toward the base material portion or a direction other than the direction toward the base material portion, is preferably acquired for the flow conductance in the space portion near a boundary between the space portion and the base material portion. In general, the flow conductance becomes isotropic regardless of the flow direction of the resin. However, in the RTM, the base material portion serves as a wall surface on which the resin flows along the boundary similarly to the inner wall surface of the mold, and also serves as a space region into which the resin can penetrate toward the inside of the base material portion. In consideration of the characteristics of the base material portion, the flow conductance that varies according to the direction toward the base material portion or the direction other than the direction toward the base material portion is provided in the space portion near the boundary such that resin flow can be more accurately analyzed.

In this case, in the space portion near the boundary between the space portion and the base material portion, the flow conductance in the direction toward the base material portion is preferably larger than the flow conductance in the direction other than the direction toward the base material portion. According to this structure, the flow conductance in the direction toward the base material portion can be prevented from being assessed to be smaller than it is in consideration of the characteristics of the base material portion into which the resin can penetrate toward the inside of the base material portion. Consequently, the influence on the resin flow in the space portion due to the characteristic penetration of the resin into the base material portion in the RTM can be properly reflected, and hence the flow analysis can be more accurately performed.

In the aforementioned structure in which the value that varies according to the flow direction of the resin, which is the direction toward the base material portion or the direction other than the direction toward the base material portion, is acquired for the flow conductance in the space portion near the boundary between the space portion and the base material portion, the acquiring of the flow conductance preferably includes computing a first conductance of each of the small elements based on the viscosity of the resin by setting the penetration coefficient as a boundary condition in the boundary between the space portion and the base material portion, and computing a second conductance based on the viscosity of the resin, assuming that the base material portion does not exist in the mold space model, and the flow conductance of each of the small elements near the boundary is preferably acquired by applying the second conductance in a case of the direction toward the base material portion and applying the first conductance in a case of the direction other than the direction toward the base material portion in the space portion near the boundary between the space portion and the base material portion. According to this structure, the second conductance is computed assuming that no base material portion exists in the mold space model such that the flow conductance in the space portion near the boundary that takes into account the penetration of the resin into the inside of the base material portion can be determined without requiring complicated computation. In addition, in the flow analysis in each of the small elements in the vicinity of the boundary, the first conductance or the second conductance is applied according to the flow direction such that the flow analysis can be more accurately performed while the amount of computation is reduced.

In the aforementioned resin flow analysis method according to the first aspect, the performing of the flow analysis preferably includes computing a pressure in each of the small elements in the mold space model based on the first relational expression and the second relational expression, computing a velocity of the resin in each of the small elements in the mold space model based on a computational result of the pressure, and computing a filled region of the resin in each of the small elements in the mold space model based on a computational result of the velocity of the resin. According to this structure, as the results of the flow analysis of the resin in the mold space, the pressure, the resin velocity, and the resin position (filled region) can be obtained. Furthermore, these analysis results can be computed based on the first relational expression and the second relational expression, and hence even when both the base material portion and the space portion exist in the RTM, analysis in a practical computation time is possible.

A non-transitory computer-readable recording medium according to a second aspect of the present invention records a program causing a computer to perform a resin flow analysis method for performing flow analysis of a resin injected into a mold using a mold space model that includes a base material portion made of continuous fiber or a porous body arranged in a sheet form and a space portion in which the base material portion is not arranged, which includes dividing the mold space model into small elements, acquiring a penetration coefficient that represents penetration characteristics of the resin into the base material portion, acquiring a flow conductance that represents flow characteristics of the resin in the space portion, and performing the flow analysis of the resin in each of the small elements in the mold space model based on a first relational expression of the small elements of the base material portion relating to the penetration coefficient, a viscosity of the resin, and a pressure and a second relational expression of the small elements of the space portion relating to the flow conductance, the viscosity of the resin, and the pressure.

The non-transitory computer-readable recording medium according to the second aspect of the present invention records the program causing the computer to perform the aforementioned resin flow analysis method for performing flow analysis of the resin injected into the mold using the mold space model that includes the base material portion made of the continuous fiber or the porous body arranged in a sheet form and the space portion in which the base material portion is not arranged, which includes the dividing of the mold space model into the small elements, the acquiring of the penetration coefficient that represents the penetration characteristics of the resin into the base material portion, the acquiring of the flow conductance that represents the flow characteristics of the resin in the space portion, and the performing of the flow analysis of the resin in each of the small elements in the mold space model based on the first relational expression of the small elements of the base material portion relating to the penetration coefficient, the viscosity of the resin, and the pressure and the second relational expression of the small elements of the space portion relating to the flow conductance, the viscosity of the resin, and the pressure. Thus, the computer is caused to read the aforementioned program and perform the resin flow analysis method such that even when both the base material portion and the space portion exist in RTM, the base material portion and the space portion can be collectively stably analyzed at a high speed while the amount of computation is reduced.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a block diagram showing a configuration example for performing a resin flow analysis method according to a first embodiment;

FIG. 2 is a schematic sectional view showing an example of a mold space model;

FIG. 3 is a diagram showing the distribution of a flow conductance;

FIG. 4 is a diagram showing examples of the shapes of small elements;

FIG. 5 is a diagram showing an example of division by the small elements of the mold space model;

FIG. 6 is a schematic view showing penetration coefficients and the distribution of a flow conductance;

FIG. 7 is a flow diagram for illustrating resin flow analysis processing according to the first embodiment;

FIG. 8 is a diagram for illustrating a first conductance according to a second embodiment;

FIG. 9 is a diagram for illustrating a second conductance according to the second embodiment;

FIG. 10 is a diagram showing an example of setting a flow conductance according to the second embodiment;

FIG. 11 is a flow diagram showing processing (subroutine) for acquiring the flow conductance according to the second embodiment; and

FIG. 12 is a graph showing the result of comparison between an analysis example based on the second embodiment and a theoretical solution.

### DESCRIPTION OF THE PREFERRED EMBODIMENTS

Embodiments of the present invention are hereinafter described with reference to the drawings.

### First Embodiment

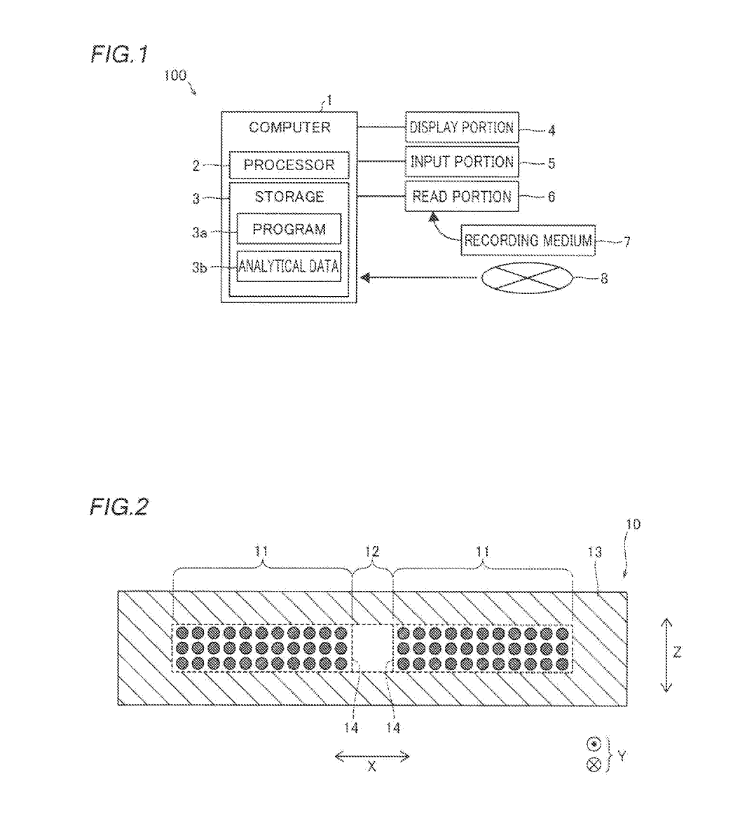

A resin flow analysis method according to a first embodiment is now described with reference to FIGS. 1 to 7.

The resin flow analysis method according to the first embodiment is an analysis method for analyzing (simulating) the flow behavior of a resin that penetrates into a base material in a mold in RTM in which a compound material is molded by arranging the base material such as continuous fiber arranged in a sheet form in the mold, injecting the resin into the mold, and causing the resin to penetrate into the base material. The base material is a fabric of carbon fiber, glass fiber, or the like, for example. The resin is a thermoplastic resin, for example. In this case, the resin is caused to penetrate into the base material in the RTM such that a molded article of fiber-reinforced plastic is molded as the compound material.

### (Device Configuration Example)

The resin flow analysis method according to the first embodiment can be performed by causing a computer 1 to run a program 3a. The resin flow analysis method can be performed by a device configuration as shown in FIG. 1, for example. The computer 1 can run the program 3a. The computer 1 is caused to run the program 3a such that a resin flow analyzer 100 is configured. Processing performed by causing the computer 1 to run the program 3a may be partially or fully performed by hardware such as a dedicated arithmetic circuit.

In a configuration example in FIG. 1, the computer 1 includes one or a plurality of processors 2 that includes a CPU (central processing unit) or the like and a storage 3 that includes a ROM (read only memory), a RAM (random access memory), a storage device, etc. The storage device is a hard disk drive, a semiconductor storage device, or the like, for example.

The computer 1 can perform resin flow analysis by causing the processor 2 to run the program 3a stored in the storage 3. The program 3a may be provided by an external server or the like through a transmission path 8 such as a network such as the Internet or a LAN (local area network) in addition to being read from a recording medium 7. The recording medium 7 is a non-transitory computer-readable recording medium such as an optical disk, a magnetic disk, or a non-volatile semiconductor memory, and records the program 3a.

The storage 3 stores various types of analytical data 3b utilized to perform resin flow analysis in addition to the program 3a. In the analytical data 3b, data of a mold space model 10 described later, numerical data (such as a penetration coefficient K) used for analysis, and data of analysis conditions such as the injection pressure of the resin into the mold, the injection flow amount, the internal pressure, and the discharge pressure are stored.

The computer 1 includes a display portion 4 such as a liquid crystal display, an input portion 5 including inputs such as a keyboard and a mouse, and a read portion 6 for reading the program 3a and the various types of data from the recording medium 7. The read portion 6 is a reader or the like according to the type of the recording medium 7. A user can input the data of the analysis conditions with the input portion 5. The analytical data 3b may be read from a recording medium prepared by the user, or may be prepared in the external server or the like by the user and be acquired from the external server through the transmission path 8.

### (Analysis Method)

Flow analysis of the resin is now described. According to the first embodiment, the flow analysis of the resin injected into a mold 13 is performed with the mold space model 10 that includes a base material portion 11 made of continuous fiber or a porous body arranged in a sheet form and a space portion 12 in which no base material portion 11 is arranged, as shown in FIG. 2. FIG. 2 is a sectional view showing an example of the mold space model 10, and schematically shows the cross-section of the inside of the mold 13 along a thickness direction (Z-axis direction). For convenience of illustration, a configuration example of a simple cross-sectional shape is shown as the mold space model 10, but the mold space model 10 actually has a spatial shape that reflects the shape of a desired molded article.

In the mold space model 10 shown in FIG. 2, the space portion 12 is arranged in a central portion inside the mold 13, and the base material portion 11 is arranged on both sides of the space portion 12. The base material portion 11 is a region in which the base material made of a fabric of the continuous fiber is arranged. FIG. 2 schematically shows the cross-section of the fiber of the base material in the base material portion 11. The space portion 12 is a region in a mold space in which no base material portion 11 is arranged. When the resin is injected into the mold space at the time of the RTM, the space portion 12 becomes a portion filled with only the resin.

The space portion 12 may be formed by intentionally forming a resin portion containing no base material in the molded article or being provided as a clearance inevitably generated when the base material is arranged in the mold 13, for example. In other words, when the base material is arranged in the mold 13 in the actual RTM, a space (clearance) may be generated between a plurality of base arranged materials, or a space (clearance) may be generated between the base material and the outer circumferential inner wall surface of the mold. FIG. 2 shows that a space is formed between one base material and another base material in the mold 13. In the resin flow analysis method according to the first embodiment, a phenomenon in which the resin simultaneously penetrates into both the base material portion 11 and the space portion 12 is collectively dealt with.

### <Base Material Portion>

Analysis of the behavior of the resin that penetrates into the base material portion 11 in the RTM is now described. The resin penetration rate of the base material portion 11 can be expressed as being proportional to the pressure gradient with the penetration coefficient based on the Darcy's law relating to the penetration of a fluid into the porous body.

More specifically, the resin penetration velocity (U, V, W) of the base material portion 11 is defined as the following expression (3) with the penetration coefficient (kx, ky, kz):

$$U = - \frac{{k}_{x}}{η} \frac{\partial P}{\partial x} V = - \frac{{k}_{y}}{η} \frac{\partial P}{\partial y} W = - \frac{{k}_{z}}{η} \frac{\partial P}{\partial z} \qquad ( 3 )$$

where x, y, and z represent three-dimensional space coordinates set in the mold space model 10, U, V, and W represent the flow rates of the resin in coordinate axis (X-axis, Y-axis, Z-axis; see FIG. 2) directions, respectively, kx, ky, and kz represent penetration coefficients in the coordinate axis directions, η represents the viscosity of the resin, and P represents a pressure. The penetration coefficient has anisotropy depending on the direction or the weave of the fiber, and hence the same is set for each of the coordinate axis directions. The penetration coefficient can be determined (actually measured) from a penetration experiment with a basic form such as a flat plate.

The following expression (4) is an equation of continuity. More specifically, the following expression (4) expresses that the sum of the inflow rate of the resin into a region of interest and the outflow rate of the resin from the region of interest is zero (the law of conservation of mass).

$$( \frac{\partial U}{\partial x} + \frac{\partial V}{\partial y} + \frac{\partial W}{\partial z} ) = 0 \qquad ( 4 )$$

The expression (3) is substituted into the expression (4) such that a first relational expression (1) is obtained.

$$\frac{K}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 \qquad ( 1 )$$

Here, K represents a penetration coefficient tensor, and is set by the penetration coefficients kx, ky, and kz in the respective directions. According to the first embodiment, the penetration coefficient K that represents the penetration characteristics of the resin into the base material portion 11 is determined in advance through the penetration experiment with respect to the base material and is stored as part of the analytical data 3 in the storage 3. The penetration coefficient K is acquired by being read from the storage 3.

The first relational expression (1) is solved such that the distribution of the pressure P of the base material portion 11 is obtained. The velocity (U, V, W) of the resin of the base material portion 11 is computed from the expression (3) with the obtained pressure distribution. Thus, according to the first embodiment, the flow analysis of the resin is performed with the first relational expression (1) of the base material portion 11 relating to the penetration coefficient K, the viscosity η of the resin, and the pressure P.

### <Space Portion>

Analysis of the behavior of the resin that flows through the space portion 12 in the RTM is now described. The above resin penetration rate of the base material portion 11 is formulated based on the Darcy's law, and the resin flow rate of the space portion 12 can also obtain a sufficient approximation by introducing the flow conductance of the space portion 12 and assuming that the resin flow rate is proportional to the pressure gradient. As a method for analyzing the behavior of the resin that flows through the space portion 12, a method detailedly disclosed in Japanese Patent Laying-Open No. 8-099341 (Japanese Patent No. 2998596) is employed, and the description of Japanese Patent Laying-Open No. 8-099341 is incorporated herein by reference.

Specifically, assuming that the resin flow velocity of the space portion 12 is proportional to the pressure gradient, the relationship between the velocity (U, V, W) of the resin and the pressure P is expressed by the following expression (5):

$$U = - \frac{c}{η} \frac{\partial P}{\partial x} V = - \frac{c}{η} \frac{\partial P}{\partial y} W = - \frac{c}{η} \frac{\partial P}{\partial z} \qquad ( 5 )$$

where c represents the flow conductance of the space portion 12. The flow conductance c represents readiness with which the resin flows through the mold space (cavity).

The above expression (5) is substituted into the equation of continuity (4) such that a second relational expression (2) is obtained.

$$\frac{c}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 \qquad ( 2 )$$

This second relational expression (2) is solved such that the distribution of the pressure P of the space portion 12 is obtained. The velocity (U, V, W) of the resin of the space portion 12 is computed from the expression (5) with the obtained pressure distribution. Thus, according to the first embodiment, the flow analysis of the resin is performed with the second relational expression (2) of the space portion 12 relating to the flow conductance c, the viscosity η of the resin, and the pressure P.

The flow conductance c that represents the flow characteristics of the resin in the space portion 12 is computed in advance prior to the computation of the second relational expression (2). When a viscous fluid flows through the space, the following expression (7) is derived by performing Stokes' approximation on the flow and assuming from the momentum conservation law that the influences of gravity and inertia are small.

$$\frac{\partial P}{\partial x} = η ( \frac{{\partial }^{2} U}{\partial {x}^{2}} + \frac{{\partial }^{2} U}{\partial {y}^{2}} + \frac{{\partial }^{2} U}{\partial {z}^{2}} ) \\ \frac{\partial P}{\partial y} = η ( \frac{{\partial }^{2} V}{\partial {x}^{2}} + \frac{{\partial }^{2} V}{\partial {y}^{2}} + \frac{{\partial }^{2} V}{\partial {z}^{2}} ) \\ \frac{\partial P}{\partial z} = η ( \frac{{\partial }^{2} W}{\partial {x}^{2}} + \frac{{\partial }^{2} W}{\partial {y}^{2}} + \frac{{\partial }^{2} W}{\partial {z}^{2}} ) \qquad ( 7 )$$

The second relational expression (2) is input into the above expression (7), and second order or higher derivative terms of x, y, and z relating to the pressure P are omitted such that the following expression (8) is obtained, noting that C₁=c/η.

$$η ( \frac{{\partial }^{2} {C}_{1}}{\partial {x}^{2}} + \frac{{\partial }^{2} {C}_{1}}{\partial {y}^{2}} + \frac{{\partial }^{2} {C}_{1}}{\partial {z}^{2}} ) = - 1 \qquad ( 8 )$$

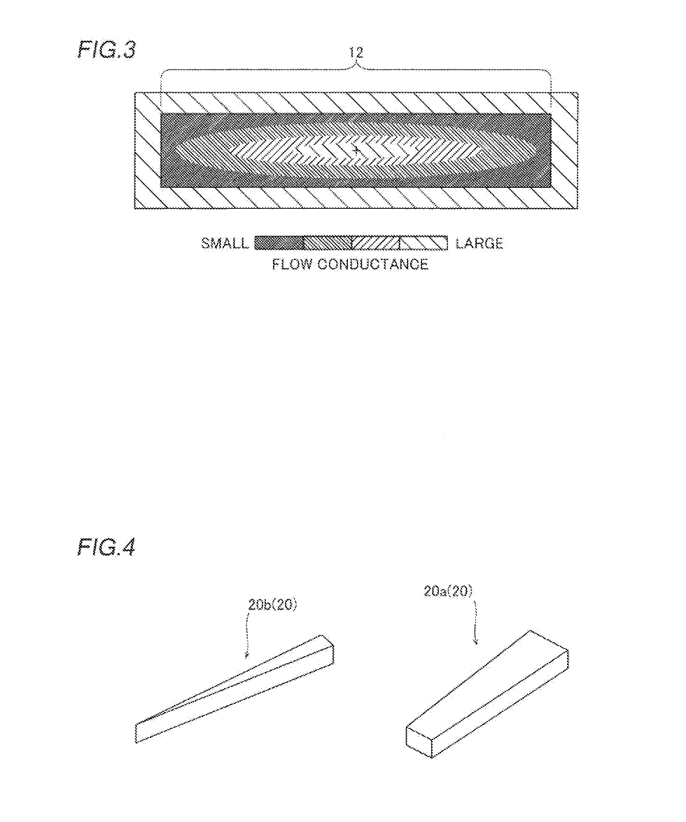

From the above expression (8), the distribution of the flow conductance c of the space portion 12 is obtained. In the obtained distribution of the flow conductance c, the flow conductance c is reduced in the outer edge (the inner wall surface of the mold) of the space portion 12, and is increased in an inner portion of the space portion 12, as shown in FIG. 3. More specifically, the flow conductance c of the space portion 12 is distributed such that the same becomes larger as a distance from the outer edge (the inner wall surface of the mold) of the space portion 12 is increased, and becomes smaller as the distance from the outer edge (the inner wall surface of the mold) of the space portion 12 is reduced.

The pressure P of the second relational expression (2) is solved with the flow conductance c computed from the above expression (8) such that the resin flow rate of the space portion 12 is determined from the above expression (5).

Thus, according to the first embodiment, the second relational expression (2) not the above expression (7), which is the Stokes' approximation formula, is used when the flow analysis for the space portion 12 is performed. Although in the above expression (7), there are four variables (U, V, W, P), in the second relational expression (2), the flow conductance c is determined such that there is one variable (P), and hence the amount of computation (computation time) is significantly reduced. The amount of computation in the three-dimensional flow analysis is proportional to the square to cube of the number of the variables, and hence according to the first embodiment using the second relational expression (2), the amount of computation is about 1/16 as compared with the above expression (7).

### <Analysis Method>

According to the first embodiment, in the mold space model 10 of the RTM including the base material portion 11 and the space portion 12, resin flow analysis in each of a plurality of small elements 20 (see FIG. 5) in the mold space model 10 is performed based on the first relational expression (1) and the second relational expression (2). More specifically, the resin flow analysis method according to the first embodiment is a method for simultaneously analyzing the space portion 12 and the base material portion 11 by collectively solving the first relational expression (1) and the second relational expression (2).

As hereinabove described, the first relational expression (1) and the second relational expression (2) are expressed by common relational expressions containing a coefficient that represents the penetration characteristics or the flow characteristics of the resin. More specifically, the first relational expression (1) and the second relational expression (2) are expressed by the common relational expressions in which coefficient parts of the penetration coefficient K and the flow conductance c are only different from each other. Thus, according to the first embodiment, the mold space model 10 is divided into the small elements 20, the penetration coefficient K is applied as the coefficient of the common relational expression for the small elements 20 of the base material portion 11 (a), and the flow conductance c is applied as the coefficient for the small elements 20 of the space portion 12 (b). Thus, the first relational expression (1) and the second relational expression (2) become similar functions, and hence the base material portion 11 and the space portion 12 can be easily continuously coupled to each other, and the space portion 12 can also be stably computed.

$$\begin{matrix}\frac{K}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 & ( 1 ) \\ \frac{c}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 & ( 2 )\end{matrix}$$

At the time of analysis, processing for dividing the space portion 12 and the base material portion 11 in the mold space model 10 into the plurality of small elements 20 as shown in FIG. 4 is first performed. As the small elements 20, a simple geometric form can be used, and a hexahedron such as a rectangular parallelepiped, a trigonal pyramid, a triangle pole, or the like is used, for example. A division operation can be performed with a publicly known a CAE (computer aided engineering) preprocessor. In division into the small elements 20, each of the space portion 12 and the base material portion 11 is divided into the small elements 20 such that in a boundary 14 (see FIG. 2) between the two, the vertices of the small elements 20 are shared.

FIG. 5 shows an example of the mold space model 10 in which the circular space portion 12 is arranged in a central portion of the annular base material portion 11. More specifically, FIG. 5 shows an example of small element division in the case where the resin is injected from the central space portion 12, and penetrates into the outer base material portion 11. In FIG. 5, the inner circumferential side of the boundary 14 shown by a bold line is the space portion 12, and the outer circumferential side of the boundary 14 is the base material portion 11. The space portion 12 and the base material portion 11 are divided into small elements 20a of the hexahedron and small elements 20b of the trigonal pyramid shown in FIG. 4, and are prepared such that their nodes are shared in the boundary 14 between the two.

Then, the penetration coefficient K measured separately is acquired, and processing for assigning the acquired penetration coefficient K is performed on the small elements 20 of the base material portion 11. The penetration coefficient K varies according to a direction in which the fiber of which the base material portion 11 is made extends, and hence the same can be set as an anisotropy penetration coefficient that varies in value for each axis direction.

Then, as to the space portion 12, the above expression (8) is solved such that the distribution of the flow conductance c of the space portion 12 is acquired. Here, the penetration coefficient K of the base material portion 11 is set as a boundary condition of the flow conductance c at the vertices of the small elements 20 in the boundary 14 between the space portion 12 and the base material portion 11, and the flow conductance c is set to zero or a value close to zero and is set as the boundary condition at the inner wall surface of the mold in order to express a no-resin slip boundary.

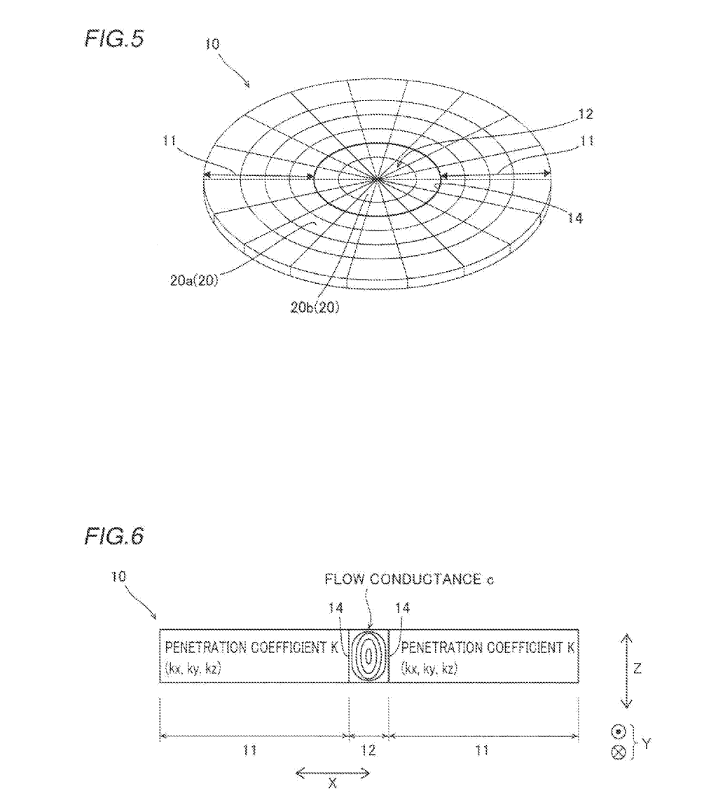

The distribution of the flow conductance c is acquired by solving the above expression (8) by the boundary condition, whereby the penetration coefficient K of the base material portion 11 and the flow conductance c of the space portion 12 are set in the small elements 20, as shown in FIG. 6. In the case of the mold space model 10 in FIG. 6, the penetration coefficient K (kx, ky, kz) is set in the outer base material portion 11, and the flow conductance c is set in the central space portion 12. The penetration coefficient K of the base material portion 11 is assigned to the boundary 14.

The data of the analysis conditions (an initial condition and the boundary condition) is set for the obtained analysis model such that numerical analysis is performed. More specifically, the injection pressure and the injection flow amount are set in a resin injection portion in the mold 13. Furthermore, a zero pressure or the discharge pressure of a corresponding portion in the mold 13 is set in the flow front of the resin.

According to the first embodiment, in processing for the flow analysis, the pressure P, the resin velocity (U, V, W), and a filled region (the position x, y, and z of the flow front) in each of the small elements 20 are computed. First, the pressure P in each of the small elements 20 in the mold space model 10 is computed based on the first relational expression (1) and the second relational expression (2). More specifically, pressure computation of the first relational expression (1) and the second relational expression (2) is performed with the data of the analysis conditions (the initial condition and the boundary condition). The pressure distribution of each of the small elements 20 of the space portion 12 and the base material portion 11 is computed. According to the first embodiment, the first relational expression (1) and the second relational expression (2) are the common relational expressions, and hence according to whether the small element 20 of interest belongs to the space portion 12 or the base material portion 11, the corresponding coefficient (the penetration coefficient K or the flow conductance c) is applied such that the common relational expression is solved.

Then, the velocity (U, V, W) of the resin in each of the small elements 20 in the mold space model 10 is computed based on the computational result of the pressure P. More specifically, resin velocity distribution in each of the small elements 20 in the mold space model 10 is computed by the above expression (3) and the above expression (5) based on the obtained pressure distribution.

Then, the filled region of the resin in each of the small elements 20 in the mold space model 10 is computed based on the computational result of the resin velocity (U, V, W). More specifically, the filled region (the position x, y, and z of the flow front) at a subsequent time step is updated based on the current velocity in the flow front.

### <Resin Flow Analysis Processing>

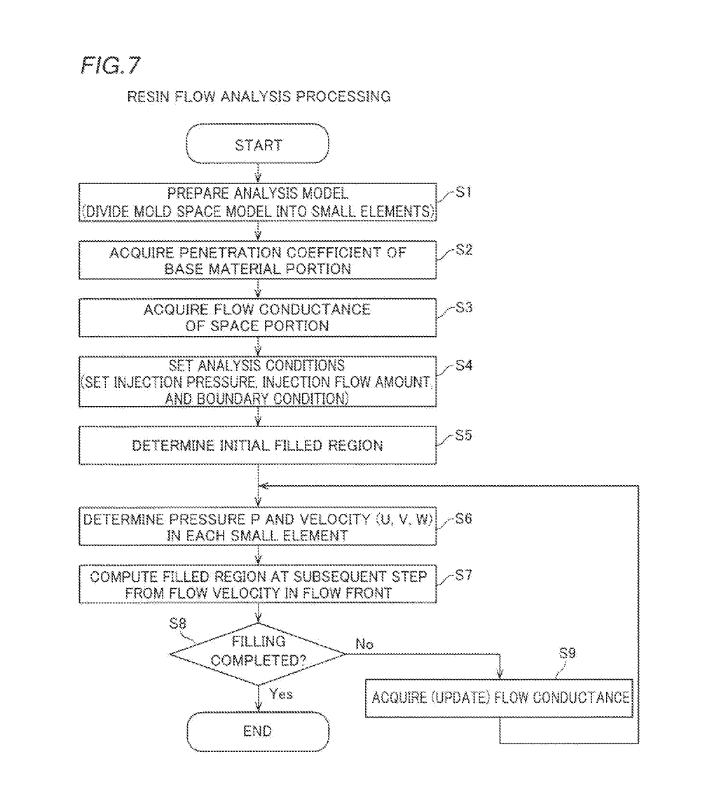

The resin flow analysis processing in the RTM is now described with reference to FIG. 7. The resin flow analysis processing is performed by the computer 1 (processor 2).

At a step S1, the computer 1 divides the mold space model 10 into the small elements 20, as shown in FIG. 5. Thus, the analysis model of the mold space is prepared.

At a step S2, the computer 1 acquires the penetration coefficient K of the base material portion 11. The penetration coefficient K is read from the analytical data 3b stored in the storage 3, for example.

At a step S3, the computer 1 acquires the flow conductance c of the space portion 12, taking the boundary condition set in advance into account. By the processing at the steps S2 and S3, the distribution of the penetration coefficient K or the flow conductance c for each small element 20 shown in FIG. 6 is set over the entire analysis model.

At a step S4, the computer 1 sets the analysis conditions. The injection pressure and the injection flow amount of the resin injection portion, the boundary condition of the flow front, etc. are set as the analysis conditions. The analysis conditions may be input through the input portion 5 by the user or may be read from the analytical data 3b stored in advance in the storage 3.

The computer 1 determines an initial (initial time step) filled region from the initial condition at a step S5, and computes the pressure P of each of the small elements 20 by the first relational expression (1) and the second relational expression (2) and computes the resin velocity (U, V, W) by the above expressions (3) and (5) at a step S6. Then, at a step S7, the computer 1 computes the filled region at the subsequent time step from the velocity in the flow front obtained at the step S6.

At a step S8, the computer 1 determines whether or not filling by the RTM is completed. When the filling is not completed, the computer 1 computes (updates) the flow conductance c at a subsequent time step at a step S9. The processing at the steps S6 and S7 is repeated such that the pressure P of each of the small elements 20, the resin velocity (U, V, W), and the filled region over time are sequentially computed. When the filling is completed at the step S8, the flow analysis is completed, and the computer 1 terminates the processing.

Thus, the computation of the pressure of each of the small elements 20, the velocity computation, and the filled region update are repeated until the filling is completed such that the resin flow analysis of the RTM is performed. The computer 1 displays the analysis results as a filling pattern showing a temporal change of the flow front, the pressure distribution, and the velocity distribution on the display portion 4. Thus, the user can determine whether the filling is good or bad, and study the effects of the shape of the molded article and/or a molding condition change by a simulation. Display of the analysis results can be performed by a postprocessor of publicly known finite element software or the like.

### Effects of First Embodiment

The effects of the first embodiment are now described.

According to the first embodiment, as hereinabove described, the resin flow analysis in each of the small elements 20 in the mold space model 10 is performed based on the first relational expression (1) of the small elements 20 of the base material portion 11 relating to the penetration coefficient K, the viscosity η of the resin, and the pressure P and the second relational expression (2) of the small elements 20 of the space portion 12 relating to the flow conductance c, the viscosity η of the resin, and the pressure P. Thus, the penetration coefficient K, the viscosity η of the resin, and the flow conductance c are acquired in advance such that the resin flow analysis can be performed on the base material portion 11 and the space portion 12 with the first relational expression (1) and the second relational expression (2), both of which use the pressure P as a common variable. The space portion 12 and the base material portion 11 can be expressed by the relational expressions having the common variable (pressure P), and hence the base material portion 11 and the space portion 12 can be collectively stably analyzed at a high speed while the amount of computation is reduced. Furthermore, the first relational expression (1) and the second relational expression (2), both of which use the pressure P as a common variable, are used such that the number of used variables can be reduced, and hence the amount of computation can be reduced. Consequently, even when both the base material portion 11 and the space portion 12 exist in the RTM, the base material portion 11 and the space portion 12 can be collectively stably analyzed at a high speed while the amount of computation is reduced.

According to the first embodiment, as hereinabove described, the first relational expression (1) and the second relational expression (2) are expressed as the common relational expressions, and at the step (S6, S7) of performing the flow analysis, the penetration coefficient K is applied as the coefficient of the common relational expression for the small elements 20 of the base material portion 11 while the flow conductance c is applied as the coefficient for the small elements 20 of the space portion 12. Thus, the base material portion 11 and the space portion 12 can be analyzed by the same relational expressions, the coefficient parts of which are different, and hence the boundary portion can be continuously dealt with, and the base material portion 11 and the space portion 12 can be collectively and stably analyzed.

According to the first embodiment, as hereinabove described, the first relational expression (1) is set as the following expression (1), and the second relational expression (2) is set as the following expression (2). Thus, the flow analysis can be performed on the base material portion 11 and the space portion 12 by the relational expressions in which all except the coefficient parts (the penetration coefficient K and the flow conductance c) are in common. Consequently, only the coefficient part (the penetration coefficient K or the flow conductance c) varies according to whether the small element 20 of interest belongs to the base material portion 11 or the space portion 12, and the entire mold space can be easily analyzed.

$$\begin{matrix}\frac{K}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 & ( 1 ) \\ \frac{c}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 & ( 2 )\end{matrix}$$

According to the first embodiment, as hereinabove described, the step (S6) of computing the pressure P in each of the small elements 20 in the mold space model 10 based on the first relational expression (1) and the second relational expression (2), the step (S6) of computing the resin velocity (U, V, W) of each of the small elements 20 in the mold space model 10 based on the computational result of the pressure P, and the step (S7) of computing the filled region of the resin in each of the small elements 20 in the mold space model 10 based on the computational result of the resin velocity are provided. Thus, as the results of the resin flow analysis in the mold space, the pressure P, the resin velocity (U, V, W), and the resin position (filled region) can be obtained. Furthermore, these analysis results can be computed based on the first relational expression (1) and the second relational expression (2), and hence even when both the base material portion 11 and the space portion 12 exist in the RTM, analysis in a practical computation time is possible.

### Second Embodiment

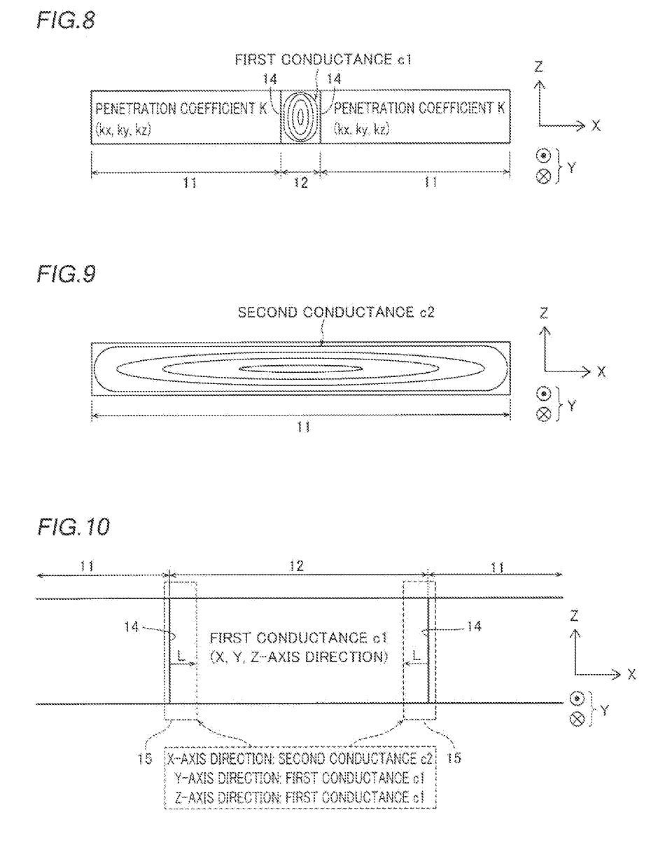

A resin flow analysis method according to a second embodiment is now described with reference to FIGS. 8 to 12. In the second embodiment, flow conductances (a first conductance and a second conductance) that vary according to the flow direction of a resin in a space portion 12 near a boundary 14 are set unlike the aforementioned first embodiment in which the single flow conductance c is set in the space portion 12.

More specifically, in the aforementioned first embodiment, the penetration coefficient K of a base material portion 11 is set as the flow conductance boundary condition of the space portion 12 in the boundary 14 between the space portion 12 and the base material portion 11 in computation of the flow conductance c of the space portion 12 (see FIG. 6). In the space portion 12, the flow conductance c of each of small elements becomes smaller as a distance to the base material portion 11 (boundary 14) becomes smaller, and the flow resistance is assessed to be large. In this case, there is no problem when the resin flows parallel to the base material portion (along the boundary 14), but the flow conductance c is assessed to be smaller than it is when the resin flows in a base material direction (X-axis direction in FIG. 6) perpendicular to a boundary surface (boundary 14) between the space portion 12 and the base material portion 11.

According to the second embodiment, at a step of acquiring the flow conductance c (steps S3 and S9 in FIG. 7), a value that varies according to the flow direction of the resin, which is a direction toward the base material portion 11 or a direction other than the direction toward the base material portion 11, is acquired for the flow conductance c in the space portion 12 near the boundary 14 between the space portion 12 and the base material portion 11. More specifically, the flow conductance c is allowed to have anisotropy in which the flow conductance c varies according to the flow direction of the resin. In the case of FIG. 6, for example, different values are set for the flow conductance c in the case where the resin flows toward the base material portion 11 in the X-axis direction perpendicular to the boundary 14 and the flow conductance c in the case where the resin flows in a Y-axis direction and a Z-axis direction along the boundary 14 other than the direction toward the base material portion 11.

Specifically, in the space portion 12 near the boundary 14 between the space portion 12 and the base material portion 11, the flow conductance in the direction toward the base material portion 11 is set to be larger than the flow conductance in the direction other than the direction toward the base material portion 11. It is only required to apply the anisotropy of the flow conductance c only in the space portion 12 near the boundary 14 between the space portion 12 and the base material portion 11. This is because at a position sufficiently away from the boundary 14, the influence on the flow conductance c from the boundary 14 is small.

As a method for setting the anisotropy of the flow conductance c, according to the second embodiment, the first conductance c1 in the direction other than the direction toward the base material portion 11 and the second conductance c2 in the direction toward the base material portion 11 are computed. Specifically, the penetration coefficient K is set as a boundary condition in the boundary 14 between the space portion 12 and the base material portion 11, the first conductance c1 of each of small elements 20 is computed based on the viscosity η of the resin, and the second conductance c2 is computed based on the viscosity η of the resin, assuming that no base material portion 11 exists in a mold space model 10.

The first conductance c1 is computed by condition setting similar to that according to the aforementioned first embodiment, as shown in FIG. 8. More specifically, the penetration coefficient K is set as the boundary condition at the vertices of the small elements 20 in the boundary 14 between the space portion 12 and the base material portion 11, zero or a value close to zero is set as the boundary condition at an inner wall surface of a mold, and the flow conductance is computed for only the space portion 12. The second conductance c2 is computed by solving the above expression (8), assuming that no base material portion 11 exists in the mold space model 10, as shown in FIG. 9. More specifically, a flow conductance computed under a condition in which the base material portion 11 is also the space portion 12 in the mold space model 10 becomes the second conductance c2. At the inner wall surface of the mold, it is only required to set zero or the value close to zero as the boundary condition. As shown in FIG. 9 (FIG. 3), the flow conductance is distributed to be larger as a distance from the boundary (the inner wall surface of the mold) is increased, and hence the second conductance c2 computed assuming that the base material portion 11 is a space is larger in value than the first conductance c1 in the vicinity of the boundary 14 (at a position corresponding to the boundary 14) due to no boundary 14.

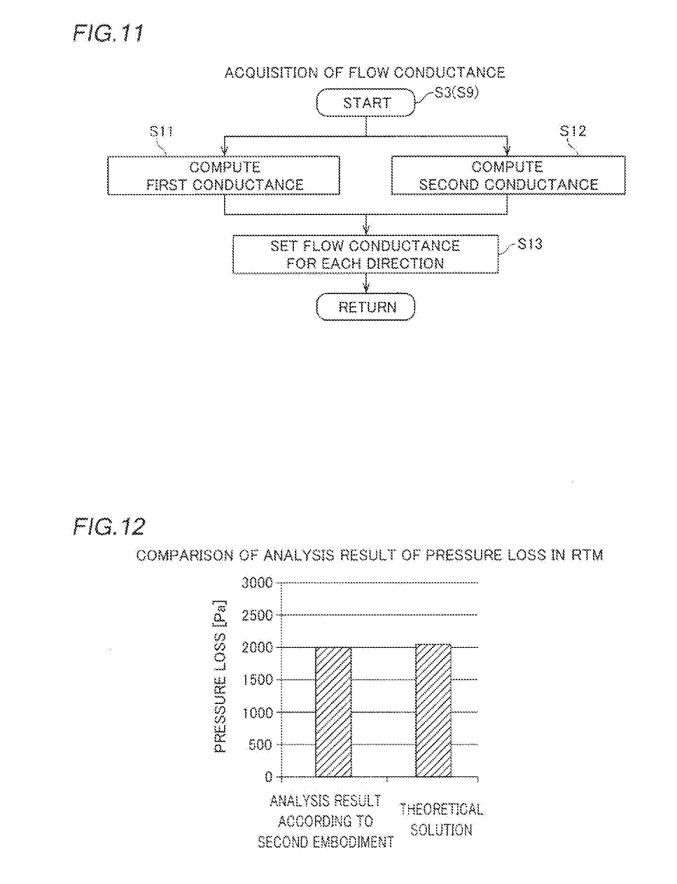

Therefore, in the resin flow analysis method according to the second embodiment, at a step S3 of computing the flow conductance c in FIG. 7, a computer 1 computes the first conductance c1 and the second conductance c2. More specifically, the computer 1 computes the first conductance c1 at a step S11, and computes the second conductance c2 at a step S12, as shown in FIG. 11. At a step S13, the computer 1 sets the distribution of the flow conductance c for each of the small elements 20.

At this time, according to the second embodiment, in the space portion 12 near the boundary 14 between the space portion 12 and the base material portion 11, the second conductance c2 is applied in the case of the direction toward the base material portion 11, and the first conductance c1 is applied in the case of the direction other than the direction toward the base material portion 11, whereby the distribution of the flow conductance c of each of the small elements 20 in the vicinity of the boundary 14 is acquired. Consequently, when the flow toward the base material portion 11 occurs in the vicinity of the boundary 14 between the space portion 12 and the base material portion 11, the second conductance c2 is used such that the flow conductance c is prevented from being assessed to be smaller than it is.

FIG. 10 shows an example of applying the second conductance c2 to a predetermined range 15 in the vicinity of the boundary 14 between the space portion 12 and the base material portion 11 on both sides of the space portion 12. In FIG. 10, the second conductance c2 is applied in the case of the X-axis direction, and the first conductance c1 is applied in the case of the Y-axis direction or the Z-axis direction in the predetermined range 15 in the vicinity of the boundary 14. In a range of the space portion 12 other than the predetermined range 15, the first conductance c1 is applied in the case of any direction.

As a method for determining the predetermined range 15 of the space portion 12 in which the second conductance c2 is applied, a method for setting a range in which a distance L from the base material portion 11 (boundary 14) is constant as the predetermined range 15 or a method for setting a range in which the influence of the base material portion 11 on the first conductance c1 is larger as the predetermined range 15 can be used.

### Effects of Second Embodiment

The effects of the second embodiment are now described.

According to the second embodiment, similarly to the aforementioned first embodiment, flow analysis is performed based on a first relational expression (1) of the base material portion 11 relating to the penetration coefficient K, the viscosity η of the resin, and the pressure P and a second relational expression (2) of the space portion 12 relating to the flow conductance c, the viscosity η of the resin, and the pressure P such that even when both the base material portion 11 and the space portion 12 exist in RTM, the base material portion 11 and the space portion 12 can be collectively stably analyzed at a high speed while the amount of computation is reduced.

According to the second embodiment, as hereinabove described, at the steps (steps S3 and S11 to S13) of acquiring the flow conductance c, the value that varies according to the flow direction of the resin, which is the direction toward the base material portion 11 or the direction other than the direction toward the base material portion 11, is acquired for the flow conductance c in the space portion 12 near the boundary 14 between the space portion 12 and the base material portion 11. Thus, the flow conductance c is provided with the anisotropy in consideration of the characteristics of the base material portion 11 that also serves as a space region into which the resin can penetrate in the RTM such that resin flow can be more accurately analyzed.

According to the second embodiment, as hereinabove described, the flow conductance c in the direction toward the base material portion 11 is made larger than the flow conductance c in the direction other than the direction toward the base material portion 11 in the space portion 12 near the boundary 14 between the space portion 12 and the base material portion 11. Thus, the flow conductance c in the direction toward the base material portion 11 can be prevented from being assessed to be smaller than it is in consideration of the characteristics of the base material portion 11 into which the resin can penetrate toward the inside of the base material portion 11. Consequently, the influence on the resin flow in the space portion 12 due to the characteristic penetration of the resin into the base material portion 11 in the RTM can be properly reflected, and hence the flow analysis can be more accurately performed.

According to the second embodiment, as hereinabove described, at the step (step S3) of acquiring the flow conductance c, the step (S11) of computing the first conductance c1 and the step (S12) of computing the second conductance c2 are provided. Furthermore, in the space portion 12 (predetermined range 15) near the boundary 14 between the space portion 12 and the base material portion 11, the second conductance c2 is applied in the case of the direction toward the base material portion 11, and the first conductance c1 is applied in the case of the direction other than the direction toward the base material portion 11 such that the flow conductance c of each of the small elements 20 in the vicinity of the boundary 14 is acquired. Thus, the second conductance c2 is computed assuming that no base material portion 11 exists in the mold space model 10 such that the flow conductance c (c2) in the space portion 12 near the boundary 14 that takes into account the penetration of the resin into the inside of the base material portion 11 can be determined without requiring complicated computation. In addition, in the flow analysis in each of the small elements in the vicinity of the boundary 14, the first conductance c1 or the second conductance c2 is applied according to the flow direction such that the flow analysis can be more accurately performed while the amount of computation is reduced.

### Analysis Example According to Second Embodiment

Comparison between the analysis result according to the second embodiment and a theoretical solution in the case where the flow conductance is computed with predetermined condition setting in the configuration example of the mold space model 10 shown in FIG. 5 is now described.

As the analysis conditions of the mold space model 10 in FIG. 5, the space portion 12 having a radius of 20 mm was arranged in the central portion, and the thickness of the mold 13 (see FIG. 2) was set to 4 mm. The penetration coefficient K of the annular base material 11 was set to 1.0×10-⁴ [mm²] in each of directions x, y, and z. Furthermore, the resin having a viscosity of 10 [Pa·s], which is a constant value, was injected with a flow rate of 10000 [mm³/sec]. A range of 10 mm between a distance of 10 mm and a distance of 20 mm from the center (the center of the space portion 12) is set as the predetermined range 15, and the analysis result and the theoretical solution of the pressure loss of the space portion 12 in the determined range 15 are compared. In the theoretical solution, the pressure loss was computed to be 2060 [Pa].

In the analysis according to the aforementioned second embodiment, the second conductance c2 in the predetermined range 15 was computed to be a value about twice larger than the first conductance c1, and as the analysis result of the pressure loss of the aforementioned predetermined range 15 in the case where the second conductance c2 is applied to the flow conductance c of the predetermined range 15, 2000 [Pa] was obtained, as shown in FIG. 12, and good agreement with the theoretical solution (2060 [Pa]) was obtained. From this, the utility of the resin flow analysis method according to the second embodiment has been confirmed.

[Modifications]

The embodiments disclosed this time must be considered as illustrative in all points and not restrictive. The range of the present invention is shown not by the above description of the embodiments but by the scope of claims for patent, and all modifications within the meaning and range equivalent to the scope of claims for patent are further included.

For example, while the example of the resin flow analysis at the time of molding the fiber-reinforced plastic molded article using the base material of the fabric of carbon fiber, glass fiber, or the like is shown in each of the aforementioned first and second embodiments, the present invention is not restricted to this. According to the present invention, the base material portion may alternatively include a base material of a porous body other than the base material of the fiber (fabric). According to the present invention, any analysis model can be applied so far as the same is an analysis model (mold space model) including a base material portion in which a base material into which a resin can penetrate in RTM is arranged and a space portion in which no base material portion is arranged, and the base material may be of any type and structure.

While the example of the mold space model having a simple circular disc shape, in which the space portion is arranged at the center and the annular base material portion is circumferentially arranged, is shown for convenience of illustration in each of the aforementioned first and second embodiments, the present invention is not restricted to this. As described above, the mold space model reflects the shape of the desired molded article, and hence the mold space model may be of any shape so far as the same includes the base material portion and the space portion. Furthermore, the shape, position, etc. of each of the base material portion and the space portion are also arbitrary.

While the expression (1) is used as the first relational expression of the base material portion 11, and the expression (2) is used as the second relational expression of the space portion 12 in each of the aforementioned first and second embodiments, the present invention is not restricted to this. The first relational expression and the second relational expression are not necessarily restricted to the expressions (1) and (2). The first relational expression can be any function relating to the penetration coefficient K, the viscosity η of the resin, the pressure P, and the second relational expression can be any function relating to the flow conductance c (c1, c2), the viscosity η of the resin, and the pressure P.

While the first relational expression (1) and the second relational expression (2) are defined as the common relational expressions (the same relational expressions in which only the coefficient parts are different from each other) containing the penetration coefficient K or the flow conductance c as the coefficient in each of the aforementioned first and second embodiments, the present invention is not restricted to this. According to the present invention, parts of the first relational expression and the second relational expression other than the coefficient parts may alternatively be different from each other.

While the flow conductance c (c1, c2) that varies according to the flow direction of the resin, which is the direction toward the base material portion 11 or the direction other than the direction toward the base material portion 11, is set (has the anisotropy) in the predetermined range 15 in the vicinity of the boundary 14 between the space portion 12 and the base material portion 11 in the aforementioned second embodiment, the present invention is not restricted to this. According to the present invention, the flow conductance may alternatively have the anisotropy over the entire space portion, for example. In other words, the flow conductance that varies according to the flow direction of the resin, which is the direction toward the base material portion 11 or the direction other than the direction toward the base material portion 11, may be set in the entire space portion beyond the predetermined range 15.

While the first conductance c1 determined for the space portion 12 by setting the penetration coefficient K as the boundary condition in the boundary 14 between the space portion 12 and the base material portion 11 and assuming the presence of the base material portion 11 and the second conductance c2 determined assuming that no base material portion 11 exists in the mold space model 10 are determined in the aforementioned second embodiment, the present invention is not restricted to this. According to the present invention, the flow conductance that varies according to the flow direction of the resin, which is the direction toward the base material portion 11 or the direction other than the direction toward the base material portion 11, may alternatively be set to a value computed by a method other than the aforementioned method for determining the first conductance and the second conductance.

While the processing operations performed by the computer are described, using the flowcharts described in a flow-driven manner in which processing is performed in order along a processing flow for the convenience of illustration in each of the aforementioned first and second embodiments, the present invention is not restricted to this. According to the present invention, the processing operations performed by the computer may alternatively be performed in an event-driven manner in which processing is performed on an event basis. In this case, the processing operations performed by the computer may be performed in a complete event-driven manner or in a combination of an event-driven manner and a flow-driven manner.

What is claimed is:

1. A resin flow analysis method for performing flow analysis of a resin injected into a mold using a mold space model that includes a base material portion made of continuous fiber or a porous body arranged in a sheet form and a space portion in which the base material portion is not arranged, comprising:
dividing the mold space model into small elements;
acquiring a penetration coefficient that represents penetration characteristics of the resin into the base material portion;
acquiring a flow conductance that represents flow characteristics of the resin in the space portion; and
performing the flow analysis of the resin in each of the small elements in the mold space model based on a first relational expression of the small elements of the base material portion relating to the penetration coefficient, a viscosity of the resin, and a pressure and a second relational expression of the small elements of the space portion relating to the flow conductance, the viscosity of the resin, and the pressure.

2. The resin flow analysis method according to claim 1, wherein
the first relational expression and the second relational expression each are expressed by a common relational expression containing a coefficient that represents the penetration characteristics or the flow characteristics of the resin, and
in the performing of the flow analysis, the penetration coefficient is applied as the coefficient of the common relational expression for the small elements of the base material portion, and the flow conductance is applied as the coefficient for the small elements of the space portion.

3. The resin flow analysis method according to claim 1, wherein
the first relational expression is a following expression (1), and
the second relational expression is a following expression (2):
$$\frac{K}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 \qquad ( 1 )$$
where K represents a penetration coefficient tensor, η represents the viscosity of the resin, x, y, and z represent positions of the small elements, and P represents a pressure of each of the small elements, and
$$\frac{c}{η} ( \frac{{\partial }^{2} P}{\partial {x}^{2}} + \frac{{\partial }^{2} P}{\partial {y}^{2}} + \frac{{\partial }^{2} P}{\partial {z}^{2}} ) = 0 \qquad ( 2 )$$
where c represents the flow conductance, η represents the viscosity of the resin, x, y, and z represent the positions of the small elements, and P represents the pressure of each of the small elements.

4. The resin flow analysis method according to claim 1, wherein
in the acquiring of the flow conductance, a value that varies according to a flow direction of the resin, which is a direction toward the base material portion or a direction other than the direction toward the base material portion, is acquired for the flow conductance in the space portion near a boundary between the space portion and the base material portion.

5. The resin flow analysis method according to claim 4, wherein
in the space portion near the boundary between the space portion and the base material portion, the flow conductance in the direction toward the base material portion is larger than the flow conductance in the direction other than the direction toward the base material portion.

6. The resin flow analysis method according to claim 4, wherein
the acquiring of the flow conductance includes:
computing a first conductance of each of the small elements based on the viscosity of the resin by setting the penetration coefficient as a boundary condition in the boundary between the space portion and the base material portion, and
computing a second conductance based on the viscosity of the resin, assuming that the base material portion does not exist in the mold space model, wherein
the flow conductance of each of the small elements near the boundary is acquired by applying the second conductance in a case of the direction toward the base material portion and applying the first conductance in a case of the direction other than the direction toward the base material portion in the space portion near the boundary between the space portion and the base material portion.

7. The resin flow analysis method according to claim 1, wherein
the performing of the flow analysis includes:
computing a pressure in each of the small elements in the mold space model based on the first relational expression and the second relational expression,
computing a velocity of the resin in each of the small elements in the mold space model based on a computational result of the pressure, and
computing a filled region of the resin in each of the small elements in the mold space model based on a computational result of the velocity of the resin.

8. A non-transitory computer-readable recording medium that records a program causing a computer to perform a resin flow analysis method for performing flow analysis of a resin injected into a mold using a mold space model that includes a base material portion made of continuous fiber or a porous body arranged in a sheet form and a space portion in which the base material portion is not arranged,
the resin flow analysis method comprising:
dividing the mold space model into small elements;
acquiring a penetration coefficient that represents penetration characteristics of the resin into the base material portion;
acquiring a flow conductance that represents flow characteristics of the resin in the space portion; and
performing the flow analysis of the resin in each of the small elements in the mold space model based on a first relational expression of the small elements of the base material portion relating to the penetration coefficient, a viscosity of the resin, and a pressure and a second relational expression of the small elements of the space portion relating to the flow conductance, the viscosity of the resin, and the pressure.
